(19) United States
(12) Patent Application Publication
(10) Pub. No.: US 2004/0191313 A1
(43) Pub. Date: Sep. 30, 2004

(54) Solid, accurately dosable pharmaceutical presentations for individual dispensing from dosing devices and methods thereof

(76) Inventors: Thomas Moest, Moorrege (DE); Klaus Lammerhirt, Moorrege (DE); Jan Ludemann, Bonningstedt (DE)

Correspondence Address: Friedrich Kueffner, 317 Madison Avenue, Suite 910, New York, NY, 10017, US

(21) Appl. No.: 10/793,674

(22) Filed: Mar. 3, 2004

(30) Foreign Application Priority Data
Mar. 5, 2003 (DE) 103 09 473.3

(51) Int. Cl. A61K 9/26; A61K 9/14

(52) U.S. Cl. 424/470; 424/489

(57) ABSTRACT

In order to provide solid, accurately dosable pharmaceutical presentations for individual dispensing from dosing devices which are, on the one hand, stable and uniformly and evenly dosable, and which, on the other hand, permit an accurate optimal individual dosage for the most different requirements by means of an accurate divisibility at will, it is proposed that the presentations constitute small spheroidal solid bodies comprising at least one active ingredient and, when needed, also pharmaceutical inactive ingredients and which have an accurate mass and active ingredient content.

The present invention relates to solid, accurately dosable pharmaceutical presentations for individual dispensing from dosing devices which constitute small spheroidal solid bodies comprising at least one active ingredient together with pharmaceutical inactive ingredients and have an accurate mass content and active ingredient content. Furthermore, the invention relates to a method for the production of the solid pharmaceutical presentations as well as their use. Such presentations facilitate an individual single dosage adapted to the requirements of an optimal treatment of patients in conjunction with dosing devices.

The production of drug forms that can be administered orally and/or perorally is known from numerous publications. As a general rule, drugs which can be administered orally are used in the form of solid preparations or liquid preparations. It is common knowledge that drugs as solid presentations, such as tablets or capsules are to be preferred to liquid preparations since they take effect in lower sections of the gastro-intestinal tract. In this way, they permit a targeted release of active ingredients enabling thereby a well controllable therapy. Moreover, solid presentations are characterised by a good stability and can be easily packed, stored and transported.

The supreme target of a drug therapy is the individual adaptation of the dosage of the active ingredients to

a) individual requirements of the individual patients and

b) the requirement of the respective phase of the therapy which the patient undergoes.

This means that a plurality of dosages of the same active ingredient are needed when patients are stabilised on certain active ingredients owing to different dose requirements, different response times or also different groups of patients (for instance adults and children). Furthermore, patients need for instance gradually increasing and gradually decreasing active ingredient dosages or quantities of active ingredients that are dependent upon the time of the day (chronopharmacology). As the case may be, the adaptation of the dose to the daily functional capacity situation of a patient is necessary. As far as the individual stabilisation of dosages is concerned, the advantage of solid presentations, such as tablets is that an accurately defined active ingredient dose is contained in an accurately separated mass that is compressed to a tablet. As a general rule, an adaptation to the individual, patient is accomplished via the size of the pharmaceutical presentation or through a division of the same.

Both possibilities of adaptation have essential disadvantages. An individual adaptation by way of the size of the tablet necessitates for each dose a new, separate licence. This is, on the one hand, expensive and, on the other hand, also unprofitable since, frequently, the minimum sales volume which is required for an individual production cannot be attained. An increase in the dose level, too, entails only a partial improvement of the patient-individual dosage since otherwise the number of most different dosage contents would rise immensely.

The divisibility of tablets for their adaptation to the patient-individual dosage does in most cases not meet the pharmaceutical requirements which are to be placed on the quality of an single-dose drug form, i.e. the uniformity of the content in relation to the separated piece.

Consequently, in the pharmaceutical market a trend towards diversification in respect of the dosage and the presentations of active ingredients has emerged caused by the rising demands on the uniformity of the active ingredient dose. As a general rule, this is, however, in actual fact not guaranteed by tablets that can be divided. The qualitative pharmaceutical deficiencies rise, due to the continuation of inaccuracy, with the number of divisions of one tablet. Therefore, tablets in their conventional use are not suited for accomplishing an easy and patient-individual dosage of active ingredients.

Liquid formulations if they are available as one-phase systems or homogeneously disperse multiple-phase systems offer in principle the possibility of the patient-individual dose adaptation by the separation of a defined volume. This is achieved by means of volumetric dosing appliances (measuring jugs) or applicators (dropper bottles). As already mentioned in the beginning, liquid presentations are, however, inferior to solid preparations. When measuring jugs are used, there exists, for instance, the problem that an inexperienced person is not able to fill a container up to the prescribed level taking into account the meniscus at the surface of the liquid. Older patients also experience substantial difficulties due to a reduced visual acuity and restrictions of motoricity. On the other hand, dropper bottles as dosage aids often cause major problems in their handling (the first drops may, for instance, not come out spontaneously; the size of the drops is wrong when the drops come out of the ventilation aperture; dependence of the size and the speed of the drops upon the way in which the dropper bottle is held, from the filling level of the bottle, from the temperature, etc.).

Since liquid drug forms should, as a general rule, possibly be made on an aqueous base in order to preclude alcohol problems in the population and e.g. to protect children, the great disadvantage is that very many active ingredients are not stable in a liquid, aqueous phase. Thus, the requirements for a patient-individual dosage can also not be adequately met by liquid forms of administration.

Consequently, the object of the present invention was to provide pharmaceutical presentations for active ingredients in such a manner that they are stable and that they can be uniformly and evenly dosed, on the one hand, and that they permit an accurate optimal individual dosage for the most different requirements through the possibility of an accurate divisibility at will, on the other hand, and to provide moreover a manufacturing and processing method.

The object of the present invention is achieved in a surprisingly easy manner by the provision of solid, accurately dosable pharmaceutical presentations for individual dispensing from dosing devices constituting small spheroidal, almost spherical or drop-shaped solid bodies.

The invention is implemented in accordance with the claims. The small spheroidal, spherical or drop-shaped solid bodies according to the invention which may also have different shapes comprise at least one active ingredient together with known customary pharmaceutical inactive ingredients and are characterised by an accurate mass content and active ingredient content. Thus they have excellent product properties and are best suited for an individual single dosage.

The particular advantage is that these small spheroidal solid bodies can be dispensed singly from relevant dosing devices, such as dosing bottles or by means of dosing spoons etc, similar to the dispensing of drops of liquid drug preparations, and that the number of solid bodies can be determined accurately when they are dispensed.

The present invention comprises moreover a method for the production of solid pharmaceutical presentations which are spheroidal or have a different shape wherein at least one active ingredient or combinations of active ingredients are converted into the desired spheroidal, almost spherical or drop-shaped pharmaceutical presentations according to well-known technologies and, if necessary, using inactive ingredients and/or additives and by means of relevant shaping tools and that thereafter the presentations thus obtained are united to a total volume defined by a specific container and that a pre-determined quantity is separated from the total volume by means of dosing devices.

The individual dosage of the spheroidal solid bodies is easy and can be accomplished by counting or by using apportioning means for the dosage. Depending upon the dose, volumetric dosing means, such as measuring jugs can preferably be used as apportioning means for a great number of required solid bodies. If only a low dose of the drug and thus a small number of solid bodies, for example less than 200 applicators are needed, a single aperture filling suggests itself for separation, such as the use of a spoon with an aperture.

According to the present invention, the spheroidal, in particular microsmall solid bodies are uniformly provided, preferably as spherical, droplike, lentil-like, almond-like or cylinder-like bodies for administration from relevant dosing devices.

In a preferred embodiment, they have a diameter of 0.5 mm to 4 mm, preferably a diameter of 1.5 mm to 3.0 mm.

The small, spheroidal solid bodies according to the invention are preferably provided for oral and/or peroral administration in the form of solid drug forms which are already known, such as capsules, coated tablets, granula, drops, globules and/or pellets.

Particularly advantageous is here the drop-shaped configuration of the solid bodies. The drop shape causes an increase in the mass in the bulgy portion of the solid bodies and thus an increase in weight within this area with the consequence that during single dispensation, e.g. from a dropper bottle the so shaped solid bodies align themselves and do not obstruct each other when they are dispensed, for example by taking up a transverse position at the dispensing opening of the dropper bottle. Furthermore, the individual solid bodies slide past each other in the direction of the dispensing opening when the dropper bottle is held in a vertical or a tilted position for the dispensing of solid bodies. For this reason, such pharmaceutical drop-shaped presentations are particularly suited since the same align themselves for counting during dispensation by means of the mass accumulated in the lower portion resulting in an additionally improved dosage possibility. Such micro-tablets have inter alia also an almost spherical shape.

The active ingredient content of an individual presentation depends upon the therapy-related dose regimen. Depending upon the active ingredient and upon its indicated field of therapy, small, solid, accurately dosable presentations are provided which make it possible to individually adapt the active ingredient to the type and scope of the therapy and to the type of patient.

The production of the solid pharmaceutical presentations according to the present invention is carried out on the basis of already known technologies using known conventional inactive ingredients and additives depending upon the input of the active ingredient and upon the desired presentation.

Also the production method and the respective form of administration of the solid bodies containing the active ingredient can be freely selected depending upon the input of the active ingredient/the active ingredients.

The production methods are, in principle, known to the person skilled in the art and they are described, e.g. in Bauer, K. N.; Froemming, K.-H.; Fuehrer, C.: Pharmaceutical Technology, Georg Thieme Publishing House, Stuttgart, 1993.

As a general rule, active ingredients (drugs) are mixed with inactive ingredients and/or additives. Thereafter, the mixtures are converted by granulation and/or pelletising into granules and/or pellets. The same are, if appropriate, processed further into tablets, capsules and coated drug forms, such as coated tablets.

The mixing of active ingredients and inactive ingredients can be done in a way that is known. The individual components can, for instance, first be mixed and then be melted open and homogenised. Depending upon the sensitivity of the active ingredient, it is recommendable to first melt open and pre-mix inactive ingredients, if appropriate, and then add the active ingredient. Mixing, melting and shaping devices which are based on a drip-off and/or forced drip-off method are known to the person skilled in the art.

Furthermore, the manufacture of spherical preparations by extrusion forming and spheronisation is, for instance, a quick method for making spherical bodies with a uniform size and shape and a smooth surface. Such spherical pellets and/or granules facilitate and improve subsequent process phases, for instance in the filling of capsules or in tableting.

The compacting of the substances for making pellets or tablets can be performed for instance by means of roller or matrix compacting. As a general rule, tableting is done in special tableting machines with the help of eccentric and rotary presses.

The spheroidal presentations according to the present invention can, therefore, as a general rule be manufactured according to all known technologies which are used for the manufacture of solid drugs, such as capsules, coated tablets, tablets, granula, drops, globules and/or pellets with the shaping of the respective presentations into spherical, drop-like, lentil-like, almond-like or cylinder-like forms being performed with the help of corresponding form tools.

The preferred presentation of the small spheroidal solid bodies in drop form is obtained by mixing the ingredients according to known methods and their shaping into drops. Using, for instance, workable inactive ingredients, a flowable mass is made in an expert manner, this mass is shaped for instance by means of rotating punching tools into drops or other spheroidal particles which are then solidified through cooling.

Such inactive ingredients and additives for the said preparations are known to the person skilled in the art. For the purposes of the present invention, all pharmaceutical substances that are safe and do not negatively react with the respective selected active ingredient are suited. Depending upon the active ingredient and upon the production method, all inactive ingredients and additives such as binding agents, filling materials, disintegrants and/or surface-active agents (wetting agents, tensides) which are customary for obtaining solid pharmaceutical presentations can be used. Moreover, all other known pharmaceutical inactive ingredients can be used.

Numerous polymer inactive ingredients are, for instance, known to the person skilled in the art, such as cellulose and cellulose, derivatives, polyvinyl pyrrolidone and/or co-polymerides which contain the same or also additives on the basis of acrylates or methacrylates.

Further components used as agents may include microdispersed silicon dioxide, carbohydrate, calcium-, magnesium- and/or glycerine stearate as well as colorants, flavourings and/or fragrances, antioxidant agents and/or stabilisers.

Their content is based on criteria which determine the mechanical-physical properties of the oral presentations, such as hardness, compressibility, size, colour and/or shape.

Drug forms as tablets or film-coated tablets can for instance be obtained by mixing the active ingredient with known inactive ingredients, such as dextrose, sugar, cellulose, lactose, sorbit, mannite, polyvinyl pyrrolidone, disintegrants such as corn starch or alginic acid, binding agents such as starch or gelatine, lubricants such as magnesium stearate or talcum and/or agents which provide a depot effect such as carboxylpolymethylene, carboxymethylcellulose, cellulose acetate phthalate or polvinyl acetate. The tablets or the film-coated tablets may also consist of several layers. Furthermore, polymers may be added or used as a film coating, such as polymethacrylic acid derivatives, cellulose acetate phthalate, HPMC, HPMC-CP, HPMC-AS. In an analogous way, coated tablets may be prepared by the coating of cores which were made in the same way as tablets with substances usually used in coatings, such as polyvinyl pyrrolidone or shellac, gum arabic, talcum, titanium dioxide or sugar. The shell of the coated tablet may consist of several layers wherein for instance the above-mentioned inactive ingredients are used. Here, too, polymers may be added or used as a film coating, such as polymethacrylic acid derivatives, cellulose acetate phthalate, HPMC, HPMC-CP, HPMC-AS. The polymers that are used here have the following function: they are entericcoated. This provides for a protection of the active ingredient from the gastric acid and/or of the stomach from the active ingredient by means of a shell and/or a film coating around the solid drops. Furthermore, the active ingredient is released in a modified manner in such a way that a prolonged therapeutic effect is obtained (retard effect). Moreover, diffusion is being controlled by means of a film membrane applied on the outside around the solid drops and by means of a matrix retardation. Substances, such as saccharin, cyclamate or sugar, and/or flavours, such as vanillin or orange extract may be added for improving the taste.

The dosage of the solid bodies formed according to the present invention is performed e.g. by means of dosing devices which make it possible to dose the solid bodies in accordance with the individual requirements in a variable quantity that can be freely chosen and, for instance, to subsequently easily control by means of markings the amount that was taken.

According to a first embodiment, a dosing device for the removal in portions of identical solid bodies by means of a sliding part encapsulated in a dispenser and actuated from the outside is characterised in that the apportioning section is filled from the inside of the dispenser and can be emptied to the outside and in that the sliding part is provided as a pipe piece comprising one or several apportioning sections and which is guided through an external cover and a stripper which is associated with the apportioning sections and is located next to the dispensing aperture with the bottom of the external cover being dome shaped.

Another dosing device is provided as a dosing spoon comprising a spoon handle and a lower section which consists of a planar polygon having an edge on all sides, with the exception of one side, and where the lower section is provided with several rows of individual indentations which are formed in such a way that one single micro-tablet fits into each individual indentation and where the individual indentations for receiving the micro-tablets in the lower section consist of cylindrical drills formed in the lower section in obliquely staggered rows and where the dosing spoon is open at its top and where the side of the lower section that has no edge runs parallel to the longitudinal axis of the spoon handle which runs in the longitudinal direction to the side of the lower section with the edge which is opposite the edge-free side of the lower section.

Further advantageous embodiments of the invention are the subject-matter of the sub-claims.

Embodiments of the invention are shown in the drawings in which:

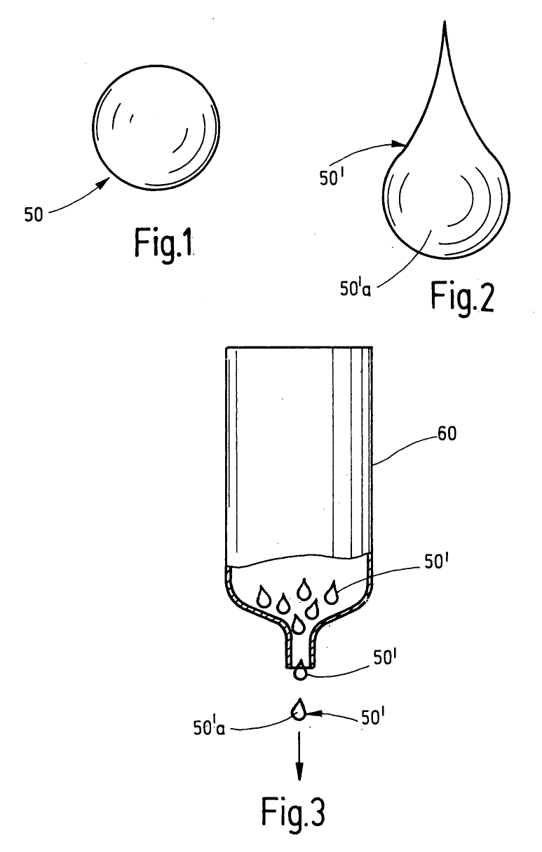

FIG. 1 and 2 are enlarged illustrations of two different embodiments of the spherical and drop-shaped solid body according to the invention;

FIG. 3 shows a dropper bottle, partly in a vertical section;

FIG. 4 shows a cross-section of a first embodiment of a dosing device for the removal of solid bodies in portions;

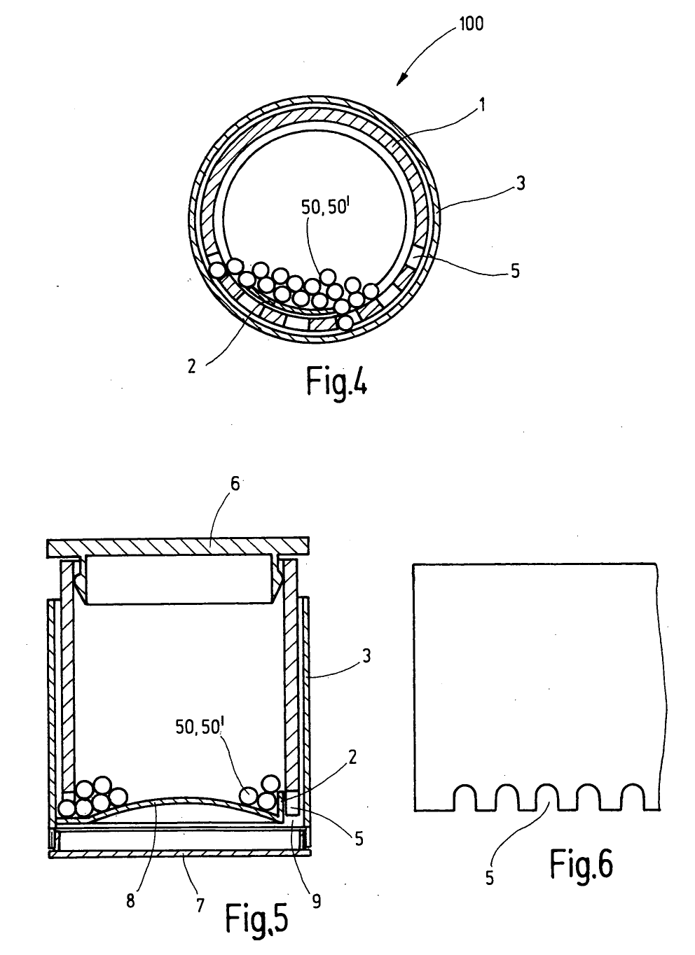

FIG. 5 shows a longitudinal section of the dosing device according to FIG. 4;

FIG. 6 shows the pipe piece of the dosing device according to FIG. 4 with a possible configuration of the apportioning section for the solid bodies;

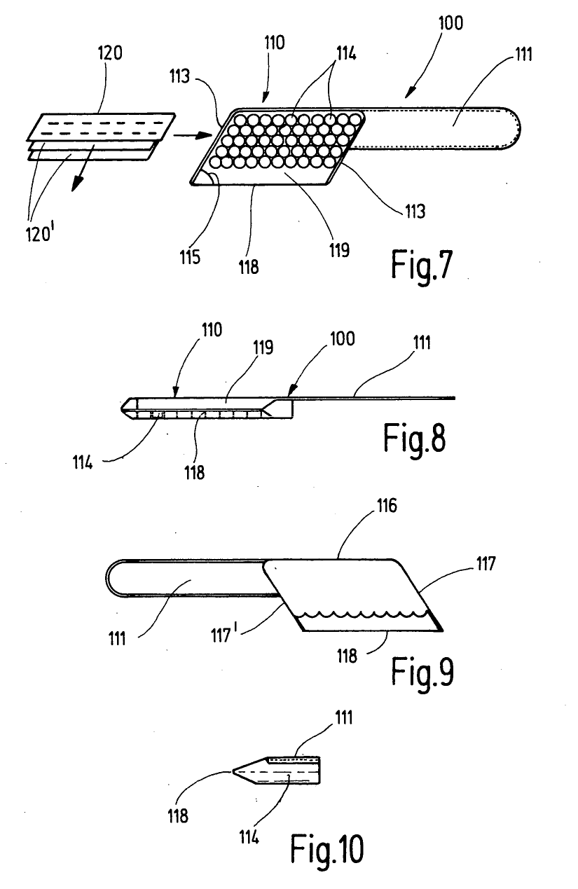

FIG. 7 shows another embodiment of a dosing device designed as a dosing spoon, in a top plan view;

FIG. 8 shows a cross-section through the dosing spoon in the longitudinal direction according to FIG. 7;

FIG. 9 shows the dosing spoon according to FIG. 7 in a view from underneath; and

FIG. 10 shows the dosing spoon according to FIG. 7 in the transverse direction.

### The solid bodies 50, 50′ designed according to the present invention have a spherical shape according to FIG. 1 and a drop-shape according to FIG. 2.

FIG. 3 shows a dropper bottle 60 for dispensing drop-shaped solid bodies 50′ which owing to their design align themselves as shown which is made possible due to the fact that the main mass of the active ingredient is located in the bulgy section 50′a.

A dispensation of solid bodies 50, 50′ in portions can also be performed by means of the dosing device 100 shown in FIGS. 4 to 10.

The dosing device 100 according to FIG. 4 comprises a dosage container which contains uniform pellets as solid bodies 50. A pipe piece 1 which is provided with apportioning sections 5 for the solid bodies is arranged between the external cover 3 and the stripper 2 of the dosage container.

The apportioning sections 5 are filled from inside the dosage container in the area which is not covered by the stripper 2 through which the solid bodies 50, 50′ get into the apportioning section 5 of pipe piece 1.

By turning the pipe piece 1 by means of an activating device 6, the respective filled apportioning section 5 is separated by stripper 2 from the interior of the dosage container and is moved to the dispensation aperture 9 in bottom 8 of the external cover 3 of the dosage container.

The dispensation aperture 9 can, for instance, be covered by a lid 7, designed as a collecting vessel. The activating device 6 serves as a cover for the interior of the dosage container. The markings for the apportioning quantities are visibly applied on pipe piece 1 from outside.

FIGS. 7 to 10 show another embodiment of a dosing device 100.

In accordance with this embodiment, the dosing device is provided as a dosing spoon 110 which is formed in such a way that the individual indentations for receiving the solid bodies in the lower section of the dosing spoon which is open at its top are cylindrical drills provided as obliquely staggered rows in the lower section and where the side of the lower section that has no edge runs parallel to the longitudinal axis of the spoon handle which runs in the longitudinal direction to the side of the lower section with the edge which is opposite the edge-free side of the lower section. Thus, the subject-matter is a dosing spoon for solid bodies with the lower section 112 of the spoon consisting of a planar polygon having an edge 113 on all sides, with the exception of one side, and where the polygon is provided with a row of individual indentations 114 which are formed in such a way that one single solid body fits into each individual indentation and/or is received therein.

As a general rule, the polygon is a square where the two opposite sides have the same length (parallelogram). The small angle 115 of the parallelogram is between 45° and 90°. One long side 116 of the parallelogram as well as the two smaller sides 117, 117′ are provided with an edge 113 which vertically projects a little bit, i.e. up to 5 mm from the polygon.

The polygon of the dosing spoon contains small cylindrical drills 114 of such diameter and depth that a solid body easily fits into each aperture. The diameter of the drills is between 1.5 and 4.0 mm. The same applies to the depth of the drills. In this special case, the diameter and the depth should be 0.2 mm greater than the longest diagonal of the micro-tablet for which the dosing spoon is to be used. The cylindrical drills are normally arranged in such a way that the greatest possible number of apertures can be provided on 1 cm² of the polygon. The total number of the apertures corresponds to the number of solid bodies that are to be taken. As a general rule, this number is 5 to 100, preferably 10 to 60.

The edge-free side 118 of the square is conveniently provided with an area without apertures 119 which normally have a width of up to 1 cm. This area facilitates the filling of the apertures with solid bodies, in particular from a container where only a few solid bodies are still left.

The spoon handle 110 is preferably attached to the longest edge and lengthens the side.

In order to be able to pre-determine the quantity and/or number of solid bodies 50, 50′ which are to be filled into the dosing spoon 110, the lower section 112 of the dosing spoon 110 can be provided with a slide 120 which is moved along one of the edges 113 in order to be able to so cover and/or close portions of the area provided with the drills 114 that, for instance, only two rows of drills 114 are available for being filled with solid bodies 50, 50′. In this way, a precise number of solid bodies can be apportioned. The said slide 120 can, for example, consist of a number of telescoping sections 120a or can be slatted (FIG. 7).

Other embodiments of the dosing devices for the solid bodies may be used, too.

The dosing devices which are described above represent preferred embodiments.

1. Solid, accurately dosable pharmaceutical presentations for individual dispensation from dosing devices where the presentations represent small spheroidal solid bodies with at least one active ingredient together with pharmaceutical inactive ingredients and have an accurate mass and active ingredient content.

2. A presentation according to claim 1,
characterised in that
the spheroidal solid bodies have a spherical, drop-like, lentil-like, almond-like or cylinder-like shape.

3. A presentation according to claim 2,
characterised in that
the spheroidal solid bodies have a drop shape.

4. A presentation according to one of claims 1 to 3,
characterised in that
the spheroidal solid bodies have a diameter of 0.5 mm to 4 mm.

5. A presentation according to claim 4,
characterised in that
the spheroidal solid bodies have a diameter of 1.5 mm to 3.0 mm.

6. A presentation according to one of claims 1 to 5,
characterised in that
the spheroidal solid bodies are provided as coated tablets, tablets, film-coated tablets, granula, drops in the solid state of aggregation and/or pellets.

7. A presentation according to one of claims 1 to 6,
characterised in that
the spheroidal solid bodies are provided with a cover made of a polymer.

8. A method for the production of solid spheroidal or differently shaped pharmaceutical presentations according to one of claims 1 to 7, characterised in that
at least one active ingredient or combinations of active ingredients is/are converted into the desired spheroidal, almost spherical or drop-shaped pharmaceutical presentations according to well-known technologies and, if necessary, using inactive ingredients and/or additives and by means of relevant shaping tools and that thereafter the presentations thus obtained are united to a total volume defined by a specific container and that a pre-determined quantity is separated from the total volume by means of dosing devices (100).

9. A method according to claim 8 for the production of drop-shaped presentations,
characterised in that
the ingredients are mixed and formed into drops in the solid state of aggregation.

10. A dosing device for removing identical presentations (4) according to one of claims 1 to 9 in portions by means of a sliding part encapsulated in a dispenser and actuated from the outside the apportioning section (5) of which is filled from the inside of the dispenser and can be emptied to the outside and where the sliding part is provided as a pipe piece (1) comprising one or several apportioning sections (5) and which is guided through an external cover (3) and a stripper (2) which is associated with the apportioning sections (5) and is located next to the dispensing aperture (9) with the bottom (8) of the external cover (3) being dome shaped.

11. A device according to claim 10,
characterised in that
the activating device (6) for the pipe piece (1) is used for closing the filling aperture.

12. A device according to claims 10 and 11,
characterised in that
a lid (7) for receiving the apportioned pellets (4) is attached to the lower section of the external cover (3).

13. A device according to claims 10 to 12,
characterised in that
markings for the apportioned quantities are provided.

14. A dosing device for removing in portions identical presentations (4) according to one of claims 1 to 9,
characterised in that
the dosing device (100) is provided as a dosing spoon (110) comprising a spoon handle (111) and a lower section (112) which consists of a planar polygon having an edge (113) on all sides, with the exception of one side, and where the lower section (112) is provided with several rows of individual indentations which are formed in such a way that one single pellet fits into each individual indentation and where the individual indentations for receiving the pellets in the lower section (112) consist of cylindrical drills (114) formed in the lower section (112) in obliquely staggered rows and where the dosing spoon is open at its top and where the side of the lower section that has no edge (118) of the lower section (112) runs parallel to the longitudinal axis of the spoon handle (111) which runs in the longitudinal direction to the side of the lower section with the edge (112) which is opposite the edge-free side (118) of the lower section (112).

15. A dosing device according to claim 14,
characterised in that
the polygon of the lower section (112) is a square in the form of a parallelogram where the opposite sides have the same length, where preferably the small angle (115) between two sides of the parallelogram is between 45° and 90° and where one long side (116) of the parallelogram as well as the two short sides (117, 117′) are provided with an edge (113).

16. A dosing device according to claims 14 and 15,
characterised in that
the edge (113) of the two shorter sides (117, 117′) of the parallelogram vertically projects a little bit, e.g. up to 5 mm from the polygon.

17. A dosing device according to one of claims 14 to 16,
characterised in that
the diameter and the depth of the cylindrical drills (114) are greater than the longest diagonal of a micro-tablet.

18. A dosing device according to one of claims 14 to 17,
characterised in that
the diameter and the depth of the cylindrical drills (114) are between 1.5 mm and 4.0 mm.

19. A dosing device according to one of claims 14 to 18,
characterised in that
the cylindrical drills (114) are arranged closely next to each other in the lower section (112).

20. A dosing device according to one of claims 14 to 19,
characterised in that
the total number of the cylindrical drills (114) in the lower section (112) corresponds to the number of the solid bodies to be received therein, the number of the cylindrical drills (114) being 5 to 100, preferably 10 to 60.

21. A dosing device according to one of claims 14 to 20,
characterised in that
the edge-free side (118) of the polygon is provided with a drill-free area, preferably of a width of up to 1 cm to facilitate the filling of the cylindrical drills with micro-capsules.

22. A dosing device according to one of claims 14 to 21,
characterised in that
portions of the drills (114) in the lower section (112) of the dosing spoon (110) can be closed by means of a slide (120) moved along one of the side edges (1 13) of the lower section (112).

23. The use of small spheroidal or drop-shaped solid bodies comprising at least one active ingredient together with pharmaceutical inactive ingredients and having an accurate mass content and active ingredient content in dosing devices (100) for an individual single dosage adapted to a patient.

24. The use according to claim 23,
characterised in that
the spheroidal solid bodies are provided in a spherical, drop-like, lentil-like, almond-like or cylinder-like shape, preferably in drop shape.

25. The use according to claim 23 or 24,
characterised in that
the spheroidal solid bodies have a diameter of 0.5 mm to 4 mm, preferably 1.5 mm to 3.0 mm.

26. The use according to one of claims 23 to 25,
characterised in that
the single dosage of the spheroidal solid bodies is obtained by counting or by using dividing means for the dosage.

27. The use according to one of claims 23 to 26,
characterised in that
depending upon the dosage level, volumetric dosing aids or dosing devices are used as dividing means for the filling of individual apertures.
